(19) United States
(12) Patent Application Publication
(10) Pub. No.: US 2025/0022579 A1
(43) Pub. Date: Jan. 16, 2025

(54) METHOD AND SYSTEM FOR CREATION AND INTERACTIVE DISPLAY OF A PRECISION HUMAN BODY BIOMAP

(71) Applicant: Cubismi, Inc., Chevy Chase, MD (US)

(72) Inventors: Moira F. SCHIEKE, Chevy Chase, MD (US); Andreas VELTEN, Chevy Chase, MD (US)

(21) Appl. No.: 18/587,314

(22) Filed: Feb. 26, 2024

Related U.S. Application Data
(63) Continuation of application No. 17/173,022, filed on Feb. 10, 2021, now abandoned.
(60) Provisional application No. 62/972,360, filed on Feb. 10, 2020.

(51) Int. Cl. G16H 30/40; G06F 3/048; G06F 18/2137

(52) U.S. Cl. CPC G16H 30/40; G06F 3/048; G06F 18/2137; G06V 2201/033

(57) ABSTRACT

The present application is directed to a system and method for creating a precision three dimensional biomap and/or digital twin representation. A representative method includes receiving, by a computing system, medical imaging data for a patient and organizing, by the computing system, the medical imaging data into hierarchical virtual space data. The method further includes creating, by the computing system, a three dimensional biomap of the patient based on the hierarchical virtual space data.

### CROSS-REFERENCES TO RELATED PATENT APPLICATIONS

This application is a continuation of U.S. patent application Ser. No. 17/173,022, filed on Feb. 10, 2021, which claims priority to U.S. Provisional Patent Application No. 62/972,360, filed on Feb. 10, 2020, the entireties of which are incorporated by reference herein.

### BACKGROUND

The following description is provided to assist the understanding of the reader. None of the information provided or references cited is admitted to be prior art.

Digital twin technology has traditionally involved the creation of a virtual copy of a real-world item (e.g., a building, object, person, manufacturing process, etc.). Digital twin technology offers tremendous promise in the area of health care and particularly personalized medicine. Prior digital twin technology in the health care space though has failed to provide sufficient detail and granularity with respect to human systems and body parts. In addition, current digital twin and image reproduction technologies suffer from imprecise registration deficiencies such that they are unable accurately translate image details across for from a patient in different positions. The foregoing application describes an improved digital twin creation technology that provides such detail and granularity utilizing a powerful machine learning system.

### SUMMARY

The foregoing summary is illustrative only and is not intended to be in any way limiting. In addition to the illustrative aspects, embodiments, and features described above, further aspects, embodiments, and features will become apparent by reference to the following drawings and the detailed description.

In accordance with one aspect of the present disclosure, a method is disclosed. The method includes receiving, by a computing system, medical imaging data for a patient and organizing, by the computing system, the medical imaging data into hierarchical virtual space data. The method further includes creating, by the computing system, a three dimensional biomap of the patient based on the hierarchical virtual space data.

In accordance with another aspect of the present disclosure, a precision biomap computing system is disclosed. The precision biomap computing system includes a database configured to store image data and a biomap creation computing unit. The biomap creation computing unit is configured to receive medical imaging data for a patient from one or more medical imaging data sources, organize the medical imaging data into hierarchical virtual space data, store the hierarchical virtual space data in the database, and create a three dimensional biomap of the patient based on the hierarchical virtual space data.

In accordance with a further aspect of the present disclosure, a non-transitory computer-readable medium having instructions stored thereon that, upon execution, cause a computing device to perform various operations is disclosed. The operations include receiving medical imaging data for a patient, organizing the medical imaging data into hierarchical virtual space data, and creating a three dimensional biomap of the patient based on the hierarchical virtual space data.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are therefore, not to be considered limiting of its scope, the disclosure will be described with additional specificity and detail through use of the accompanying drawings.

FIG. 1 depicts a representation of a biomap creation and display system in accordance with an illustrative embodiment.

FIG. 2 depicts a block diagram representing various components associated with the biomap creation system of FIG. 1 in accordance with an illustrative embodiment.

FIG. 3 depicts an example flow diagram outlining a method for creating a digital twin biomap in accordance with an illustrative embodiment.

FIG. 4 depicts an example flow diagram outlining a method for converting real space data to hierarchical virtual space data in accordance with an illustrative embodiment.

FIG. 5 depicts an example flow diagram outlining a method for creating a three-dimensional (3D) precision biomap in accordance with an illustrative embodiment.

FIG. 6 depicts a visual representation of the process of converting raw medical imaging data into a precision digital twin biomap in accordance with an illustrative embodiment.

FIG. 7 depicts a flow diagram for a method of mapping data according to a biomechanical model in accordance with an illustrative embodiment.

FIG. 8 depicts an example flow diagram outlining an alternative method 800 for creating and/or updating a three-dimensional (3D) precision biomap in accordance with an illustrative embodiment.

FIG. 9 depicts a visual representation of a mapping of super-resolution grid coordinates in 3DEXTCAR in accordance with an illustrative embodiment.

FIG. 10 provides a visual representation of image data segregated into voxels and mapped to the anatomy of a patient in accordance with an illustrative embodiment.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

Precision medicine is a medical model that proposes the customization of healthcare practices by creating advancements in disease treatments and prevention. The precision medicine model takes into account individual variability in genes, environment, and lifestyle for each person. Additionally, precision model often uses diagnostic testing for selecting appropriate and optimal therapies based on a patient's genetic content or other molecular or cellular analysis. Advances in precision medicine using medical images may be further bolstered by a powerful imaging platform as described herein that creates and displays a digital twin (virtual replication) of an individual. Creation and display of such a digital twin may be aided through collection and analysis of big data.

Further, big data may be leveraged to create valuable new applications for a new era of precision medicine. Image volumes generated from an individual patient during a single scanning session continues to increase, seemingly exponentially. Multi-parameter MRI can generate a multitude of indices on tissue biology within a single scanning session lasting only a few minutes. These new powerful systems using big data form the basis for identification and deployment of new imaging and analysis techniques.

Specifically, big data offers tools that may facilitate identification of the new imaging biomarkers. Big data represents information assets characterized by such a high volume, velocity, and variety to require specific technology and analytical methods for its transformation into value. Big data is used to describe a wide range of concepts: from the technological ability to store, aggregate, and process data, to the cultural shift that is pervasively invading business and society, both drowning in information overload.

Big data coupled with machine learning methods may be used to obtain super resolution images that facilitate identification of the new imaging biomarkers. In particular, machine learning methods, such as classifiers, may be applied to the images of the subject to output probabilities for specific imaging biomarkers and/or other tissue characteristics, such as normal anatomy and correlation to pathology tissue data (herein also defined as image biomarkers) based on comparisons of features in sets of the images of the subject and population-based datasets and big data that provide similar information, but for other subjects. By applying the machine learning methods, high or super resolution images may be obtained that may then be used for identifying and/or measuring the biomarkers.

However, tracking precision biomarkers over time requires precision mapping of medical imaging data, other biomedical and clinical data, at a granular level across a plurality time-points—but current methods do not meet these needs. State-of-the-art image registration methods only allow registration of 2D to 2D or 3D to 3D volumes of imaging data. In addition, complex motion and non-uniform positioning of the body during scanning severely limits the precision of these registrations. For example, the liver is known to compress by approximately 30%, with variance in the amount of compression internally versus peripherally, and based on factors affecting organ stiffness, such as cirrhosis. Obtaining two imaging slices at two separate timepoints at the exact anatomical location is not currently possible. In addition, a person's body morphology can change over time, such as from new lesions or tissue, such as cancer, cysts, or fat, or from normal losses, such as seen with age-related osteoporosis. Precision tracking of imaging and other data about a patient's body over time, and secondary biomarkers which can be obtained from this data, is currently not possible. The techniques described within enable precision mapping of medical imaging data via an anatomical coordinate system regardless of the position of the patient during imaging or time delays between images.

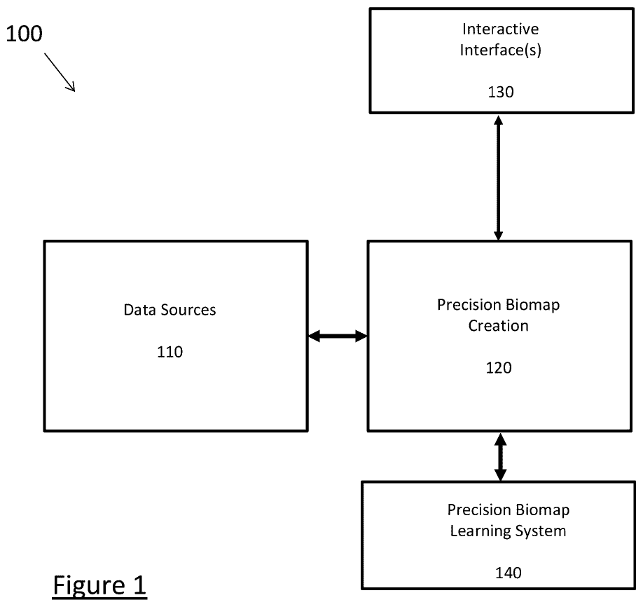

FIG. 1 depicts a representation of a biomap creation system 100 in accordance with an illustrative embodiment. The biomap creation system 100 includes one or more data sources 110, a precision biomap creation component 120, interactive interfaces 130, and a precision biomap learning system 140. In an embodiment, the biomap creation system 100 and/or its constituent components may include one or more computing devices or processing units configured to execute instructions. The instructions may be carried out by a special purpose computer, logic circuits, or hardware circuits. The processing units may be implemented in hardware, firmware, software, or any combination thereof. The term “execution” is, for example, the process of running an application or the carrying out of the operation called for by an instruction. The instructions may be written using one or more programming language, scripting language, assembly language, etc. The precision biomap creation component 120, the interactive interface 130, and the precision biomap learning system 140, thus, may execute an instruction, meaning that they perform the operations called for by that instruction. In some embodiments, the precision biomap creation component 120, the interactive interface 130, and the precision biomap learning system 140 may be implemented in separate computing devices or systems (including the cloud) that are communicatively networked together. In other embodiments, the precision biomap creation component 120, the interactive interface 130, and the precision biomap learning system 140 may be implemented together in a single computing device or together across multiple computing devices (e.g., in the cloud or across other networked devices).

The one or more data sources 110 may include any data source for gathering patient data relevant for the creation of a precision biomap and digital twin for the patient. In an embodiment, the one or more data sources 110 may include a medical imaging apparatus (e.g., magnetic resonance imaging (MRI), computed tomography (CT), positron emission tomography (PET), ultrasound, etc., equipment), sensor data, lab data equipment (e.g., genetics mapping equipment), medical equipment, electronic health care records, patient information (submitted for example by a user interface or other suitable mechanism), or any other data source capable of conveying patient data. In an embodiment focused on incorporating medical image data, the patient data gathered from the one or more data sources 110 may include one or more of molecular and/or structural imaging data such as MRI parameters, CT parameters, PET parameters, single-photon emission computed tomography (SPECT) parameters, micro-PET parameters, micro-SPECT parameters, Raman parameters, bioluminescent optical (BLO) parameters, and ultrasound parameters. Additional, external photographic images or other types of surface images of the patient's body may be gathered.

The interactive interface(s) 130 provide an interactive graphical user interface by which a user may interact with a 3D representation of a person's body map digital twin. The interactive interface(s) 130 include a display depicting the body map digital twin as well as various navigation tools to allow a user to implement any of numerous navigational instructions including, e.g., select a point region or segment/region (such as an organ, such as the liver) on the 3D body; selecting a point or segment/region via axial, coronal, and sagittal images obtained from medical imaging equipment that are mapped to the person's biomap; allowing a user to “dive” into the tissue at a specific location in order to “zoom” to a selected higher resolution. The interactive interface(s) 130 may also allow selection of data from the precision biomap system and/or associated database for precision analytics utilizing machine learning algorithms to compare patient data to population data. In this way the interactive interface(s) 130 together with other components of the precision biomap system can be used to determine biomarkers for the patient and for use as a digital twin for interrogation and use of such data for modelling of data for clinical questions and predictions. Further, enabled by cloud technology, multiple interfaces can operate collectively to enable interactions with multiple users across a geographically diverse system of computers.

The interactive interface 130 enables elements and specific views of the human body depicted in the digital twin to be shared across various systems, apps, displays, etc., in an ecosystem, such as a healthcare system. For example, a 3D virtual representation of a patient's body selected by a radiologist on his or her interface can be shared in apps for the ordering physician and patients for clear communication and real-time coordination for optimal clinical care. The 3D body interface may also allow a user to annotate the depiction of the digital twin with descriptions and tags to highlight specific segments/regions or point locations of a patient's body to be saved back into the database. In addition, the interactive interface(s) 130 allow a user to easily pull out all images related to a specific feature (e.g., a dermatologist may pull out all images over time for a skin lesion).

The biomap creation component 120 converts user data received from the one or more data sources 110 into a precision 3D biomap that may be used to create the body map digital twin display via the interactive interface 130.

Precision biomap learning system 140 updates the 3D biomap models of patients' bodies over time as additional data becomes available for the patients. In an embodiment, precision biomap learning system receives new data from the data sources 110 and performs updates to existing models based on an analysis of the new data in a similar manner as the biomap creation component 120 creates the original precision 3D biomap.

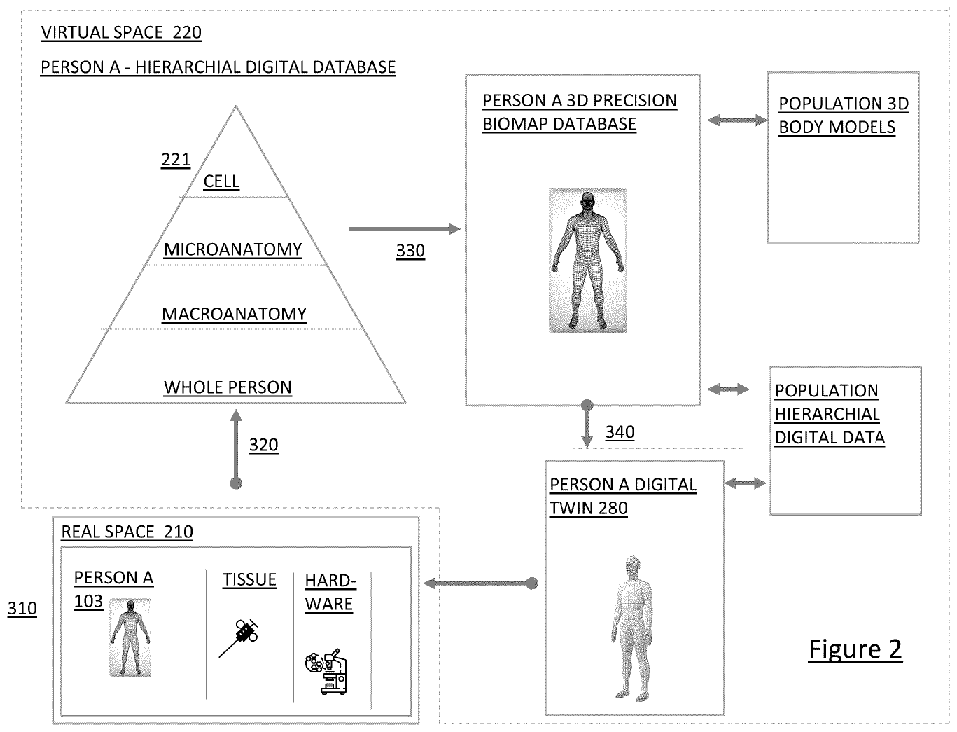

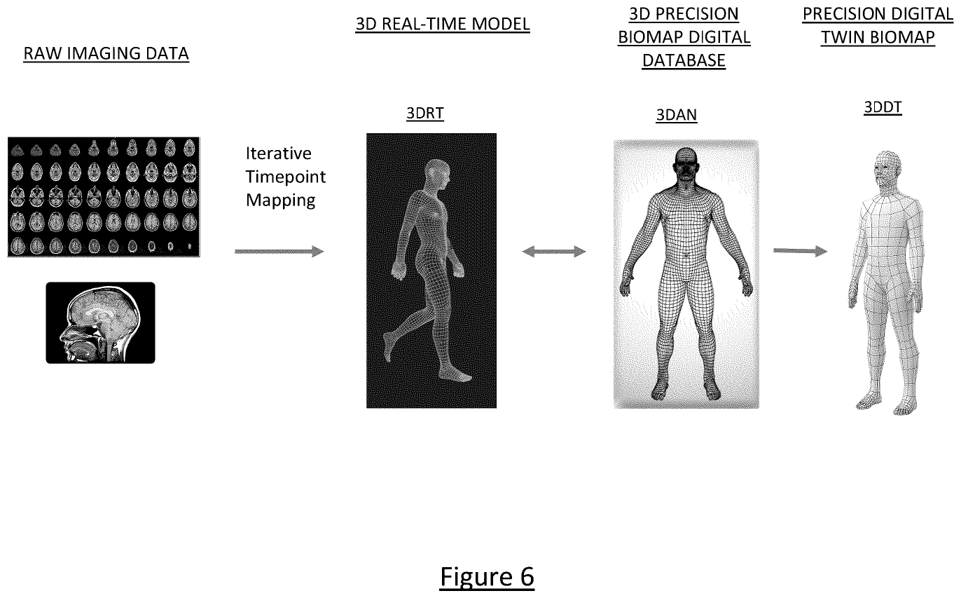

FIG. 2 depicts a block diagram representing various components associated with the biomap creation system 100 of FIG. 1 in accordance with an illustrative embodiment. FIG. 6 depicts a visual representation of the process of converting raw medical imaging data into a precision digital twin biomap in accordance with an illustrative embodiment. As depicted in FIG. 6, raw imaging data is mapped to a respective 3D real-time (3DRT) model associated with a first timepoint. Additional iterative mappings of imaging data to additional, respective real-time models may be performed for imaging data associated with other timepoints. Image from the various 3DRT models across various timepoints are further mapped to a 3D anatomically neutral (3DAN) model. The 3DAN model may be used to create a biomap digital twin interface.

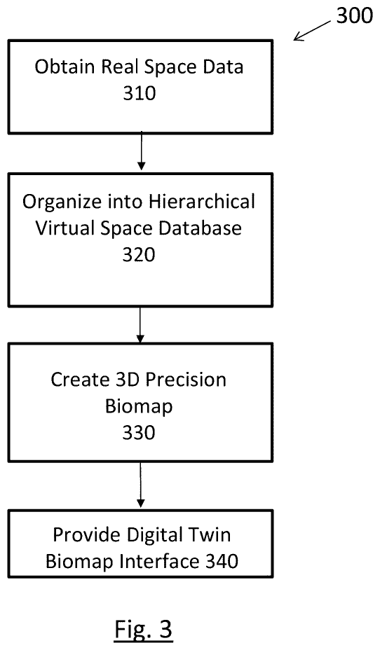

FIG. 3 depicts an example flow diagram outlining a method 300 for creating a digital twin biomap in accordance with an illustrative embodiment. In an embodiment, method 300 may utilize the components of the biomap creation system 100 depicted in FIGS. 1 and 2.

In an operation 310, real space data is obtained by the biomap creation system 100. As depicted in FIG. 2, real space 210 may be a patient 103, a body of the patient 103, objects on or within the body of the patient (e.g., surgical hardware, tubes, sensors, etc.), tissue from the patient's body, external hardware, lab equipment used to assess the patient (e.g., for genetic testing), medical images, etc. Accordingly, real space data may be data associated with any of data sources 110. In addition, real space data may include data entered by health care personnel, electronic health record information, diagnoses, etc. In an embodiment, real space data includes medical imaging data and is obtained via imaging devices (MRI, CT, PET, digitized pathology slide, etc.) that are communicatively coupled to other components of the biomap creation system such as the biomap creation component 120.

In an operation 320, the real space data is converted into hierarchical virtual space data 221. For example, the real space data is mapped by a hierarchical data system 120 into the virtual space 200 and organized into a hierarchical data structure 221. Each instance of real space data is assigned a timepoint representative of the time at which the instance of real space data was obtained.

In an embodiment, the real space data comprises medical image data that is segregated into a plurality of voxels. Each voxel represents a three-dimensional volume within the patient. In addition, each voxel is annotated with a plurality of features associated with respective hierarchically distinct portions of the anatomy of the patient. For example, each voxel may be assigned a first feature associated with a cell-level corresponding to the portion of the patient represented by the voxel, a second feature associated with a microanatomy level for the voxel, a third feature corresponding to a macroanatomy level for the voxel, and a fourth feature corresponding to a name of the patient. Accordingly, each voxel is saved with a plurality of metadata including one or more of an intensity value, a position value (e.g., an x, y, and z position or other coordinate system positions/values), a voxel volume value, and one or more anatomy feature labels of varying hierarchical levels.

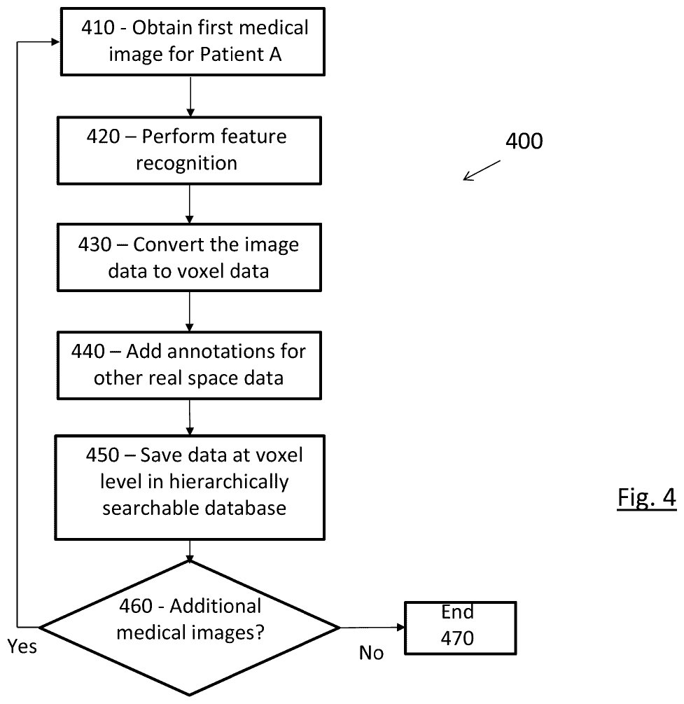

FIG. 4 depicts an example flow diagram outlining a method 400 for converting real space data to hierarchical virtual space data in accordance with an illustrative embodiment. Method 400 includes operation 410 in which medical image data is obtained for Patient A. In an embodiment, the medical image data may include one or more MRI images of a patient. In alternative embodiments, the medical image data may include any other type of medical image data as well as other types of imaging data including data regarding patient surface information from photographs, a millimeter wave scanner, or other suitable imaging equipment.

In an operation 420, features are identified in the medical image or a surface image. In an embodiment, features refer to components of the patient's anatomy or other non-anatomical objects within a patient's body (e.g., hardware, foreign bodies, etc.). For example, a feature may be a cell, a group of cells, an organ, a part of an organ, a region of the body, a cyst, a tumor, etc. A feature may also be a specific and distinctive location in or on a person's body such as an internal corner of an eye. Still further, a feature can be a descriptive non-anatomical parameter such as a volume of a voxel, a biomechanical tensor, or biomarker data from classifications comparing population data.

In an embodiment, to identify features in the medical image or surface image, a moving window of various sizes is moved across the image as described for example in U.S. Pat. No. 10,776,963, which is incorporated herein by reference. In each window, the window content is compared to a database of existing features. In response to a determination of sufficient similarity between the window content and that of a known existing feature, the system flags the corresponding feature and associates it with the portion of the image corresponding to the position of the moving window.

The algorithm is also trained to find the locations and outlines of features in the human body. In an example embodiment, a convolutional neural network is trained using a dataset of labeled training images. In another embodiment, the voxels in a moving window in the patient (or pixels in photographs of the patient) may be converted into a feature vector. The feature vector could be computed using manually chosen operations, like Fourier transform or moments, or features could be learned as a dictionary, for example using a principal component analysis or independent component analysis. After computing the features, the vector is classified using, for example, a support vector machine that has been trained on features from a labeled image dataset.

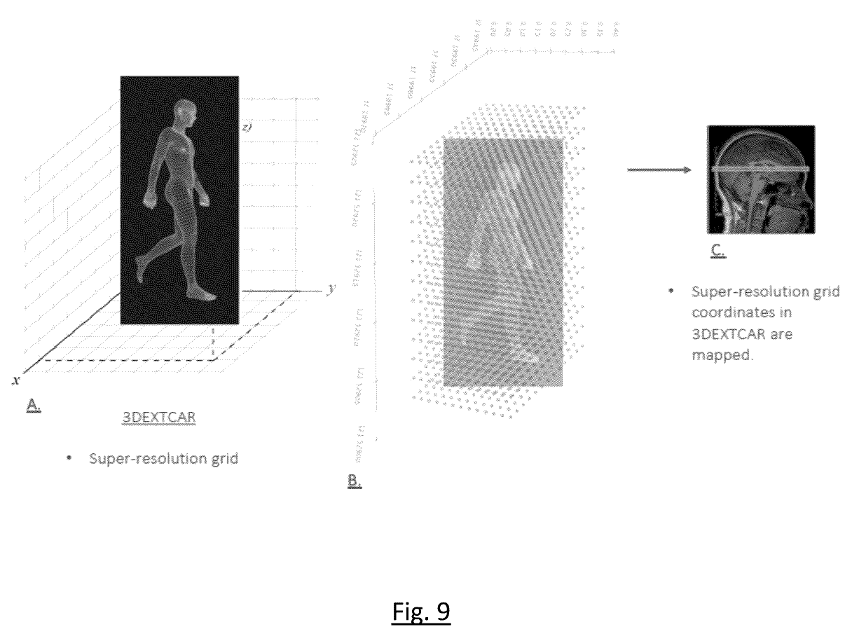

In an operation 430, the medical image data is segregated into voxels that each represent a volume within the patient. Each voxel includes an identifier indicating its position relative to other voxels and/or other portions of the patient in three dimensional space. There is a known relationship between the external cartesian coordinate system around a patient during imaging and the imaging coil or other equipment around the patient that collects the medical imaging data. Accordingly, based on machine settings, it is precisely known where the image slices are obtained for a person's body relative to the external cartesian coordinate system. Thus, as an example, an axial slice of the brain may be obtained and the exact cartesian coordinates of the slice determined in 3D volume external (3DEXTCAR), with a voxel size smaller than that of standard medical images MRI resolution of 1 mm in width, and as low as the size of a single cell or less. This is graphically illustrated in FIG. 9 which visually depicts mapping of super-resolution grid coordinates in 3DEXTCAR. Segregation of the medical image data (e.g., imaging slices) may involve sequential processing of multiple consecutive imaging slices, where each slice is of a location contiguous to the prior slice.

The specific size of the volume associated with the voxel may be modified based on a desired resolution of the associated biomap to be created from the voxel information. For example, voxel size can range from relatively large (resulting in sparse labelling most suitable for macroanatomy) to very dense labelling (enabling detailed labelling down to the cellular level (e.g., on the order of 100 micrometers)). Each voxel is annotated with the one or more features identified in operation 420, such that the one or more features describe the specific anatomy of the patient corresponding to the volume represented by the respective voxel. In an embodiment, the voxel may be annotated with multiple features in a hierarchical fashion such that the features described various hierarchical levels of the anatomy. As an example, a voxel may be annotated with features describing the voxel as being associated with the portal vein (first hierarchical level), liver (second hierarchical level), abdomen (third hierarchical level), etc.

In an embodiment, the voxel is annotated using a neural network and a dataset of trained images to compare the image data associated with the voxel (and possibly surrounding or nearby voxels) to a population database of anatomically annotated voxels. Anatomy feature labels can be obtained from resource atlases such as the National Library of Medicine human body project, the HUBMAP cell atlas project, or other naming conventions known to those of skill in the art.

In alternative embodiments, a segmentation technique can be used to identify and label components across multiple voxels. Segmentation can be achieved by grouping pixels with similar values. Alternatively feature vectors can be computed from moving windows in the image using, for example, a principle component analysis and matching these feature vector to a set of features from a learned dictionary. Segmentation and labelling can be achieved using structure or modality specific contrast in the images. Machine learning algorithms may be used to identify resulting features and label points or regions of the anatomy including classification techniques such as support vector machines, structure labels, and convolution neural networks. Alternatively, segmentation can be performed using a diffeomorphic mapping between the unknown image (i.e., the medical image being analyzed) and a large database of known images of respective previously identified and structures.

In an operation 440, a voxel may be annotated with additional features aside from anatomically descriptive features. For example, a voxel may be annotated with timestamp information, patient identification information, diagnosis information, genetics data from a tissue biopsy, physical annotations, electronic health record information, etc. Such features may include any information that may eventually be helpful in the creation of or interaction with a digital twin biomap (including determination of image biomarkers). For example, such features could include biomechanical data such as tensor information for liver stiffness quantification. In addition, medical imaging data from other sequences or image types obtained at the same slice location and same timepoint, such as using a PET-MRI machine, can be mapped to an anatomically-correct image. For example, low resolution and warped data from PET images and diffusion-weighted images (DWI) can be registered using standard techniques to anatomically-correct T1 spin echo images from the same slice location and time, and each voxel in the anatomically correct T1 spin echo image can be updated with PET and DWI uptake and signal data. Further, other data associated with the voxel can be annotated, including imaging biomarkers, biomechanical data, and all other potential types of analytics data outputs. Further, in additional to 2D images, 3D imaging may be obtained from medical images using standard techniques and all voxels in the 3D image stack can be similarly mapped.

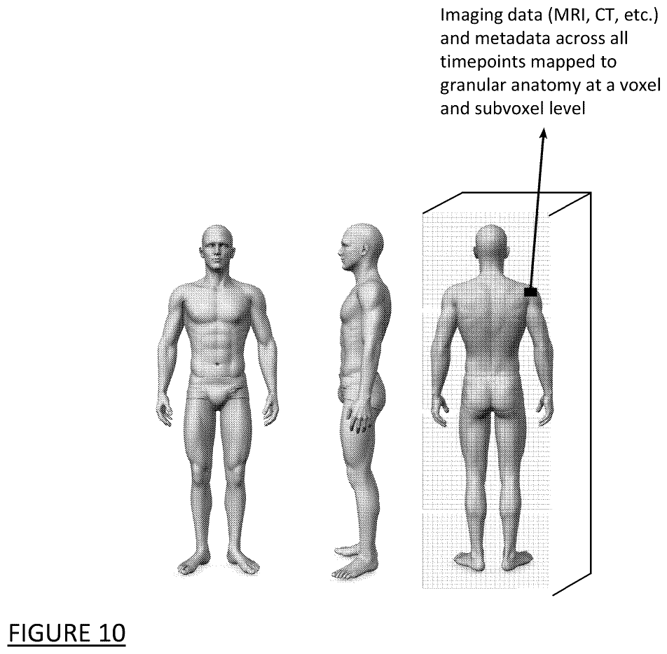

In an operation 450, the feature and source data annotations are saved for each voxel in a searchable hierarchical database such that the voxels can be selectively search by feature annotation. In an embodiment, each voxel is saved together with searchable metadata that identifies a specific feature associated with each of a plurality of hierarchical anatomical levels within the body. FIG. 10 provides a visual representation of image data segregated into voxels and mapped to the anatomy of a patient.

In an operation 460, if additional medical images or other types of images (e.g., surface images) are available for analysis for the patient, the method returns to step 410 and processes the additional medical images in the same manner. In one embodiment, the additional medical images may be a same type of medical image (e.g., MRI) maybe taken at different timepoints. In other embodiments, the additional medical images may be a different type of medical image (e.g., PET vs. MRI vs. CT) that provide new data not available in prior analyzed medical images. If no additional medical images are available, the method 400 ends at operation 470.

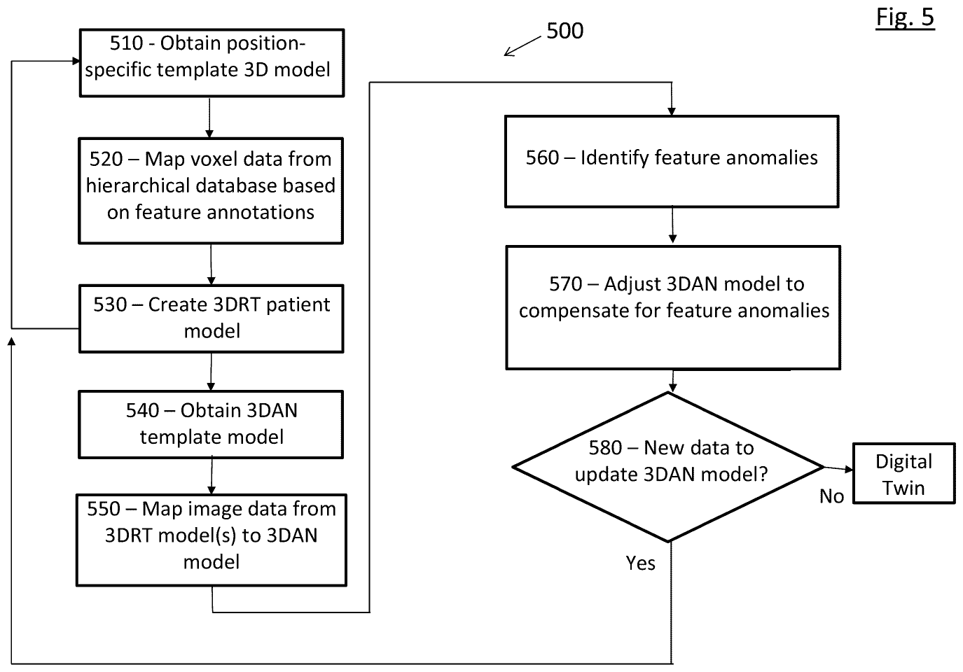

In operation 330 of FIG. 3, a 3D precision biomap is created from the hierarchical virtual space data created in operation 320. FIG. 5 depicts an example flow diagram outlining a method 500 for creating the 3D precision biomap in accordance with an illustrative embodiment. Method 500 includes operation 510 in which a position-specific template 3D model is obtained for a first timepoint (e.g., timepoint 1). Different template models may be selected from a library of templates for different genders, body types, etc., and modified based on characteristics of a specific patient. The position-specific (and also time-specific) template 3D model is a template 3D model for the patient that corresponds to a specific external position and internal organ position (and compression) of the patient during the taking of a particular medical image at a specific time. The template model has an associated external cartesian space composed of rectangular voxels at a selected voxel dimension and resolution (see, e.g., 3DEXTCAR in FIG. 9); voxel volumes may range in volume be the approximate volume of an entire organ, an MRI voxel (1 mm), or cell on a pathology image of tissue (1 um). The position-specific template 3D model defines the surface area and contours of the patient in the specific position. In an embodiment, the position-specific surface data for creating the template 3D model may be created for the patient based on one or more photographic (or other) images taken of the patient in the corresponding position. Feature points from the images are mapped to a chosen template model from a population of templates models of human movement (across a variety of races, gender, BMI, etc.) and the template model is morphed to create a 3D model of the patient's body in its real-time position (i.e., position at the current timepoint) both internally and externally. In alternative embodiments, the position-specific template 3D model may be selected from a database of template models by comparing medical image data for the patient taken while the patient is in the corresponding position to medical image data associated with the various template models. According to such techniques, a library of highly detailed 3D body models of the human body across a population will be created with standardized demarcations of established anatomy with voxel-based codes for volumes have small volumes (e.g., measuring approximately 1 mm or smaller). The library of 3D models of the human body will range across a variety of body shapes, sizes, sexes, races, etc., across the entire global population. This library is used to train the feature recognition algorithms. The anatomy in the models is organized in an “out to in” organization such that the body outlines encompass the organ segmentation, which outlines the sub-organ anatomy, which outlines smaller anatomy ranging in size down to the level of a single cell or smaller. In an embodiment, the anatomy may be described to the level of an organelle. In another particular example embodiment, a number of voxels are contained within the outline segmenting the liver. A single 1 mm voxel within the segmented liver volume is further segmented into smaller voxels, measuring roughly 1/20th mm. According to this embodiment, microanatomy is mapped within these smaller voxels (1/20th mm).

In an embodiment, the position-specific template 3D model may be modified based on current information about the patient (e.g., height, weight, body mass index, age, sex, etc.) prior to further processing or mapping of the voxel data as described below.

In an operation 520, all voxel data associated with a same timepoint (or timestamp) obtained in the process of FIG. 4 is mapped to corresponding voxels in the position-specific template 3D model. For example, the various hierarchical feature data for each voxel is mapped to corresponding voxels in the position-specific template 3D model. Alternatively, landmark rigid registration methods can be used to update the feature data for each voxel without modifying image values. Image data mapping may start with the most anatomically-correct imaging data, such as CT data and T1 spin echo data. In addition, to the hierarchical feature data regarding the corresponding anatomy of the voxel, additional data including timestamp information, patient identification information, diagnosis information, electronic health record information, etc., may be mapped to the voxel in the template model. Voxelwise data will be mapped to the 3D model at a set threshold of volume matching of the input image data voxel volume and the 3D model 3DEXTCAR voxel volume. For example, the data may be mapped only when the 3D model 3DEXTCAR voxel volume fits 100%, 90%, or 30% (or any other selected threshold) within the volume of the mapped input image voxel. When criteria for voxel matching are met, all feature, source data, metadata, and other data associated with the input image voxel can be mapped, or a subset of the feature, source data, and metadata may be mapped.

Upon mapping of all voxel data associated with a same timepoint to the position-specific template 3D model, a three-dimension real-time model is created for the patient for the specific timepoint. The three-dimension real-time model comprises a plurality of voxels each representing specific volumes within the patient at that timepoint. Each voxel will include one or more hierarchical feature annotations describing the anatomy within the volume represented by the voxel, as well as source data, metadata, and other data, such as biomechanical data and biomarker data.

In the event additional voxel data for images associated with the patient at a different timepoint is available, the method may return to operation 510 and operations 510-530 may be repeated to create a second 3DRT patient model associated with the second timepoint (and a potentially second, different position for the patient). In this way, the process may be continued to map all timepoints for which imaging data is available.

In an operation 540, a 3D anatomically neutral (3DAN) template model or existing 3DAN model already containing the patient's data from a prior timepoint is obtained for the patient. The 3DAN template model is a template model for the patient that corresponds to an anatomically neutral position that is consistent across all patients for purposes of creation of the biomap. For example, in one embodiment the anatomically neutral position could correspond to a person in a standing position with 30 degree angles at the shoulders and 20 degree angles at the hips, with knees straight and without any valgus or varus angulation. In other embodiments, an alternative position may be chosen. The 3DAN template model defines the surface area and contours of the patient in the anatomically neutral position as well as the corresponding normal associated position of internal organs and extent of organ compression. The “normal” characteristics associated with the 3DAN template model may be based on a population database or population-wide standard. For example, the template model could be generated based on a consensus average for the anatomies and feature locations of all (or of a representative sample) of prior patients. In an embodiment, 3DAN template model may be selected from a database of template models by comparing information about the patient (e.g., sex, height, weight, age, etc.) to corresponding information from the database of template models. Different template models may be provided for different genders, body types, etc. and modified based on characteristics of a specific patient. For example, the position-specific template 3D model may be further modified after selection based on information about the patient (e.g., height, weight, age, sex, etc.) prior to further processing or mapping of the voxel data as described below.

Similar to the 3DRT template, the anatomically-neutral template model has an associated external cartesian space composed of rectangular voxels at a selected voxel dimensions and resolution; voxel volumes may range in volume be the approximate volume of an entire organ, an MRI voxel (1 mm), or cell on a pathology image of tissue (1 um). According to such techniques, a library of highly detailed 3D body models of the human body across a population will be created with standardized demarcations of established anatomy with voxel-based codes for volumes have small volumes (e.g., measuring approximately 1 mm or smaller). The library of 3D models of the human body will range across a variety of body shapes, sizes, sexes, races, etc., across the entire global population. This library is used to train the feature recognition algorithms. The anatomy in the models is organized in an hierarchical “out to in” organization such that the body outlines encompass the organ segmentation, which outlines the sub-organ anatomy, which outlines smaller anatomy ranging in size down to the level of a single cell or smaller. In an embodiment, the anatomy may be described to the level of an organelle. In another particular example embodiment, a number of voxels are contained within the outline segmenting the liver. A single 1 mm voxel within the segmented liver volume is further segmented into smaller voxels, measuring roughly 1/20th mm. According to this embodiment, microanatomy is mapped within these smaller voxels (1/20th mm).

In an operation 550, image data including features from one or more 3DRT models is mapped to the 3DAN template model. In an embodiment, the features and other data associated with each voxel in the 3DRT model are mapped to an associated voxel in the 3DAN template model. This mapping amounts to a geometric transform to convert from a Cartesian coordinate system (3DEXTCAR) of the 3DRT model to an anatomical coordinate system of the 3DAN template model, which in turn is associated with another external cartesian coordinate system (3DEXTCAR2). Mapping of the voxels of the 3DRT model to the 3DAN template model may utilize one or a combination of several possible geometric transform conversion methodologies including 1) a biomechanical model; 2) a motion model; and 3) big data sets.

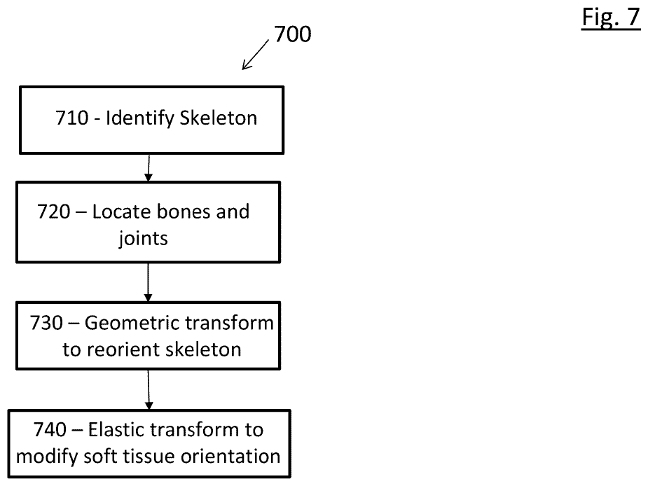

The biomechanical model uses a decision-tree model that leverages biomechanical information about various tissues, joints, and range of motion to predict behavior of various tissues (including level of compression, stretch, deformation, etc.) at different positions (e.g., lying on a scanner bed vs. standing). FIG. 7 depicts a flow diagram for a method 700 of mapping data according to such a biomechanical model. In an operation 710, a segmentation technique is used to identify bones from the medical images. In an embodiment, classification of the bones may be automated based on contrast from CT or MRI images and a machine learning method trained on an image database or may be manual inputs from a physician based on review of the medical image. Each voxel is labelled with various information including as an example tensor information related to anatomy feature labels.

In an operation 720, location of joints and evaluation of the angles between bones at the joints is performed to determine the extent of the geometric transform required to move the bones/joints to the predefined anatomically neutral position. In an embodiment, the central axis of each analyzed bone is determined and the intersection of the axes of bones at a joint is determined and the joint angle (i.e., the angle between the bones of the joint) is computed. The required adjustment of this current joint angle to meet the anatomically neutral joint angle is then determined.

In an operation 730, a geometric transform is performed to change the current joint angle to the anatomically neutral joint angle based on the calculations in the foregoing step. In this way, the skeleton is reoriented to the anatomically neutral position by turning or repositioning the rigid bones at the joints, adjusting the angles of the bones, and/or otherwise moving the position(s) of bones in the skeleton.

In an operation 740, an elastic transform is performed to modify the shape and position of the soft tissue around the bones and adjust the position of the internal organs such that the 3DRT data is also moved to the anatomically-neutral model. Each region of anatomy in the model carries a defined tensor and carries information about how the tissue relates to surrounding tissues to determine the exact motion of the 3DRT data. For example, a beating heart causes compression on adjacent organs and structures, but other tissues may slide past each other. The boundary of the tissue is defined at the surface of adjacent, contiguous bones. Points between the boundaries established by the ones are transformed to an anatomically neutral position using a transformation model. In an embodiment, the transformation model uses a 3D linear interpolation technique to re-mesh all points between bone attachment surfaces. In alternative embodiments, machine-learning based techniques may be used.

Alternatively, a motion model using known relationships between a human body in many potential positions versus the predefined anatomically neutral position may be used. Further, a machine-based learning technique leveraging neural networks (or other techniques) trainer on large datasets may be used to transform the positioning of the body.

In an embodiment, a given 3D body is chosen from the library of 3D models that best matches the patient undergoing medical imaging. All voxel data are labeled with exact anatomical topological locations of human anatomy and/or variant human anatomy in accordance with the patient imaging data and the selected model.

During the geometric transform from 3DRT to 3DAN, the 3DEXTCAR2 voxels associated with the 3DRT model are morphed. In order to re-generate rectangular voxels, the voxels in the external cartesian coordinate system (3DEXTCAR2) associated with 3DAN are updated with the transformed data; annotations, feature labels, source data and other data mapped from the 3DRT data to the 3DAN 3DEXTCAR2 voxels. Further, the mapping of data to the 3DAN model is thresholded at a set level of volume matching between input 3DRT voxel volumes and the 3D model 3DEXTCAR1 voxel volumes. For example, the 3DRT voxel data may be mapped only when the 3DAN 3DEXTCAR2 voxel volume fits 100%, 90%, or 30% (or any other selected threshold) within the volume of the mapped morphed input image voxel. When criteria for voxel matching are met, all feature, source data, metadata, and other data associated with the input image voxel can be mapped, or a subset of the feature, source data, and metadata may be mapped.

In an embodiment, only features (such as anatomical labels) considered to meet a threshold level of confidence are used to determine which data is mapped from the 3DRT model(s) to the 3DAN template model and which features are used in the biomechanical and other methods for the geometric transform. The degree of confidence in the feature point is in most cases directly provided by the feature recognition algorithm. For example in a support vector machine classifier one would obtain the quality of match to a particular feature as a distance in feature space. In a neural network, the network would be designed such that it provides a confidence score along with the classification result. One could also investigate how stable the classification is under perturbation of the input data. Alternatively, proximity could be used to determine the confidence score. If a feature that is supposed to be located in the liver is located far away from the liver, the confidence score for the feature would be very low confidence. In subsequent iterations, the proximity measure would become more and more stringent. As the alignment between the image and the prototype becomes better, features would be expected to become very close to where they should be based on the template model. Further, confidence in feature labels could be determined by the source data. Surface data obtained at a given time-point would provide higher confidence features about the patient's body than modeled internal organ data in the absence of associated medical imaging data. Confidence value data would be stored with other voxel data. Further, each time a new 3DRT is mapped to the 3DAN model holding patient data from prior timepoints, the 3DAN morphology would be updated based on mapped new 3DRT data that reflect the updated morphology of the patient based on high confidence voxels. Examples interval changes in patient morphology of existing tissue, such compression, or could be a new feature anomaly, such as a lost limb, a tumor growth, new hardware, etc.

In an operation 560, feature anomalies are identified in one or both of the modified 3DAN template model and the 3DRT model(s). Anomalies may include orphaned features (e.g., features in the modified 3DAN template model that do not appear in the 3DRT model(s)) or void regions (e.g., regions where there is tissue in the 3DRT model(s) but not in the modified 3DAN template model). An orphaned feature may be due to the growth of a cyst or tumor, due to foreign bodies, due to an injury or loss of tissue due to normal aging, or due to the presence of any other unexpected object. Void regions may appears as unusually large empty spaces without corresponding features.

In an operation 570, the 3DAN template model is further adjusted to compensate for the identified feature anomalies. In an embodiment, the regions (e.g., groups of voxels) associated with the feature anomalies are segmented and additional feature recognition performed to identify the specific feature (e.g., cyst, tumor, injury, etc.) that is the cause of the anomaly. Alternatively, such anomalies may be identified by user inspection conducted via a user interface on which the model and anomalies are presented. The 3DAN template model is then modified and morphed such that the feature portions are changed and/or new features and regions are assigned based on correct data about the patient. Accordingly, when a feature point in the real space image is matched to a feature point in the 3DAN model the location of the feature point in the 3DAN model is changed to match the location of the matched point in the real space dataset. The region around the feature point in the 3DAN model is adjusted using an elastic transform such that all other matched feature points maintain their positions and only unmatched regions of the 3DAN model can move. As a result unmatched features in the real space dataset will move closer to their counterparts in the 3DAN model and have a higher confidence and a higher change of being matched based on proximity in a next iteration of the algorithm.

In an operation 580, the system checks to determine if new data (e.g., a new 3DRT model) is available to update the 3DAN model. If it is, the operation returns to operation 510 and the subsequent operations are repeated. It no data is not available, the 3DAN model made available for presentation as a digital twin via a corresponding user interface.

In an operation 340, a precision digital twin biomap is created based on the 3D precision biomap. The digital twin biomap is presented on a graphical user interface and enables a user to perform a variety of actions to answer specific clinical questions about the patient. Such actions may include review of a detailed reproduction of the patient's body, selection of a subset of data such as identification of data and biomarkers for precision analytics, conversion between multiple views/angles/models, analysis of and response to clinical questions, diagnoses, communication to other platforms or interfaces, etc.

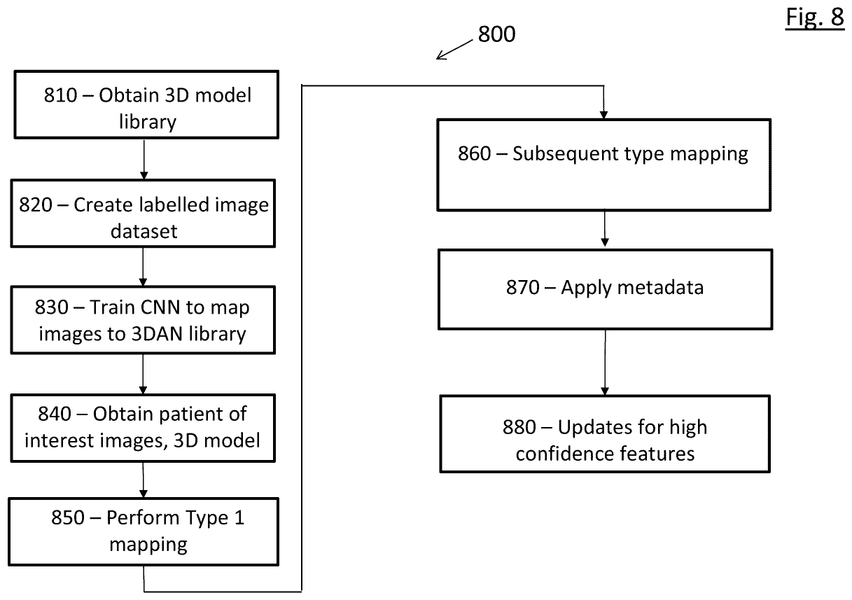

FIG. 8 depicts an example flow diagram outlining an alternative method 800 for mapping input imaging data (and all associated voxel data) to the 3D models, including the template 3D models for the 3DRT and 3DAN, as well as the 3DRT and 3DAN containing patient data in accordance with an illustrative embodiment. In an embodiment, the method of flow FIG. 8 provides an alternative pathway for the method and/or similar methods to map the voxel data from hierarchical database (520 in FIG. 5) based on feature annotations to the 3DRT models (530 in FIG. 5) or alternately map the images directly to the 3DAN template model or 3DAN. Features may refer to any of the possible features discussed elsewhere in this disclosure. In various embodiments, the features may be a single feature common to both input images and a 3D model (e.g., anatomy labels), the features could be multiple labels (e.g., multiple anatomy feature labels for macroanatomy and microanatomy), or a combination of feature data and other data such as tensor data and source image data. This alternative mapping strategy would be employed only when high confidence features are labelled in both the input images and the corresponding 3D model. Further, this alternative pathway could only be employed when the 3D body model reflects the current morphology of the patient's body. This pathway may be particularly applicable where the model data may be more accurate than the source data, such as with anatomically morphed DWI MR images.

As described in FIG. 8, previously described methods can be used for the selection of various 3DRT or 3DAN template models or 3DRT or 3DAN models as required elsewhere in this disclosure. In such an instance a CNN or other neural network may be used to directly map new 2D and 3D data to 3D body models by registering annotated voxelwise and pixelwise (for surface data obtained from photographic images) feature labels in the source data image and 3D model.

In an operation 810, a 3D model of choice (template, 3DRT, or 3DAN) is selected as required elsewhere in this disclosure. At block 820, anatomical labels are created on a separate population of patient 2D or 3D images as required elsewhere in this disclosure. At block 830, a CNN system is trained to map 2D or 3D images to a large population of 3D body models (which could include templates, 3DRT, or 3DAN). A CNN may be used to train the system to map selected features (such as anatomical features) coded in both the voxels of the input 2D or 3D images and the 3D body models using standard CNN such as a ResNet or an AlexNet. Subsets of the population could be used for training, for example, only women, only men, etc. Direct regression may be used with an “hourglass network” CNN architecture, an extension of the fully convolutional network, using skip connections and residual learning, or another type of CNN architecture. The result is a 3D registration of the source image to the 3D body models in 3D space with a resulting warped deformation field in 3D. The level of precision of the mapping would depend of the volume of data available for training, the confidence level of the feature labels in the images and 3D models, and the number of features and number of voxels containing those features in both the input images and 3D models.

At block 840, input 2D or 3D images from a patient of interest are gathered and the voxel features are labeled for these images. Alternately, the input data may be the source data used to construct the image, such as attenuation data or K-space data. These images may be acquired from a scanner and may provide a K-space value for at least one of a given volume and a reconstructed image volume. K-space value is associated with at least one of a given volume and a reconstructed image volume from MRI, or attenuation data from CT, X-rays, or other primary imaging data from other types of scanners. The K-space data may be alternately directly mapped to 3D models.

At block 850, input 2D or 3D medical images and photographs from the patient of interest of a first type (Type 1) are mapped to the selected 3D model type using the trained CNN. At block 860, images from the patient of other types (Types 2, 3, 4 . . . ) are mapped to the selected 3D model type using the trained CNN using the associated trained CNN. The new 3D model can have a very high resolution 3DEXTCAR1 or 3DEXTCAR2 voxel grid with each voxel measuring less than 1 mm, and potentially smaller than 1 micrometer. At block 870, source data, metadata, and other data associated with mapped voxels are filled into the new 3D model at each voxel. The source data, metadata, and other data may include but is not limited to all the following types of data (up to 1,000's, 100,000's, and 1 million data points): 1) imaging value 1; 2) imaging value 2; 3) parameter map 1; 4) parameter map 2; 5) ML algorithm 1; 5) ML algorithm 2; 6) imaging value 1, timepoint 2; and 7) anatomical feature labels. At 880, feature labels in the original 3D model are updated only when the input image data anatomical feature data is higher confidence than the original 3D model feature labels. For example, new external photographs containing high confidence features of the patient's face may be used to update the 3D model and provide a 3D model with close resemblance to the patient, for graphical display as a digital twin on the user interface.

As discussed above, the biomap creation system 100 and its components may include one or more processing units configured to execute instructions. The instructions may be carried out by a special purpose computer, logic circuits, or hardware circuits. The processing units may be implemented in hardware, firmware, software, or any combination thereof. The term “execution” is, for example, the process of running an application or the carrying out of the operation called for by an instruction. The instructions may be written using one or more programming language, scripting language, assembly language, etc. The biomap creation system and its components, thus, execute an instruction, meaning that they perform the operations called for by that instruction.

The processing units may be operably coupled to the one or more databases for storage and analysis of various patient data (including hierarchical anatomical feature data) as well as voxel data for precision biomaps and digital twin interfaces. The biomap creation system 100 and its components may retrieve a set of instructions from a memory unit and may include a permanent memory device like a read only memory (ROM) device. The components may copy the instructions in an executable form to a temporary memory device that is generally some form of random access memory (RAM). Further, the biomap creation system 100 and/or its various components may include a single stand-alone processing unit, or a plurality of processing units that use the same or different processing technology.

With respect to the hierarchical digital database, the precision biomap database and other databases discussed herein, those databases may be configured as one or more storage units having a variety of types of memory devices. For example, in some embodiments, these databases may include, but not limited to, any type of RAM, ROM, flash memory, magnetic storage devices (e.g., hard disk, floppy disk, magnetic strips, etc.), optical disks (e.g., compact disk (CD), digital versatile disk (DVD), etc.), smart cards, solid state devices, etc. The interactive interfaces 130 may be provided on an output unit, which may be any of a variety of output interfaces, such as printer, color display, a cathode-ray tube (CRT), a liquid crystal display (LCD), a plasma display, an organic light-emitting diode (OLED) display, etc. Likewise, information may be entered into the biomap creation system 100 and its components (including data sources 110) using any of a variety of unit mechanisms including, for example, keyboard, joystick, mouse, voice, etc.

Furthermore, only certain aspects and components of the biomap creation system 100 are shown herein. In other embodiments, additional, fewer, or different components may be provided within the system.

It is to be understood that although the present disclosure has been discussed with respect to cancer imaging, the present disclosure may be applied for obtaining imaging for other diseases as well. Likewise, the present disclosure may be applicable to non-medical applications, particularly where detailed super-resolution imagery is needed or desired to be obtained.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (for example,, “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (for example, the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. A method comprising:
receiving, by a computing system, medical imaging data for a patient;
organizing, by the computing system, the medical imaging data into hierarchical virtual space data; and
creating, by the computing system, a three dimensional biomap of the patient based on the hierarchical virtual space data.

2. The method of claim 1, further comprising converting the three dimensional bimap to a digital twin biomap interface and providing the digital twin biomap interface for display via a graphical user interface.

3. The method of claim 1, wherein creating the three dimensional biomap comprises:
converting the medical imaging data to a first real-time three dimensional model corresponding to a first timepoint;
converting second medical imaging data to a second real-time three dimensional mode corresponding to a second timepoint; and
creating a three dimensional anatomically neutral model based on the first and second real-time three dimensional models.

4. The method of claim 1, wherein organizing the medical imaging data into hierarchical virtual space data comprise:
performing a feature recognition algorithm to identify features of the patient based on the medical imaging data; and
converting the medical imaging to voxel data, wherein the voxel data include the identified features.

5. The method of claim 4, further comprising saving the voxel data in a hierarchically searchable database.

6. The method of claim 1, wherein creating the three dimensional biomap of the patient comprises:
obtaining a first position-specific template 3D model corresponding to a position of the patient when the medical imaging data was obtained for the patient;
mapping voxel data associated with a first timepoint from the hierarchical virtual space data into the first position-specific template; and
in response to the mapping, creating a first three-dimensional real-time model of the patient.

7. The method of claim 6, wherein creating the three dimensional biomap of the patient further comprises:
obtaining a three-dimensional anatomically neutral template model; and
mapping image data from the first three-dimensional real-time model to the three-dimensional anatomically neutral template model to create a patient-specific three-dimensional anatomically neutral biomap.

8. The method of claim 7, further comprising:
identifying feature anomalies in the patient-specific three-dimensional anatomically neutral biomap; and
adjusting the patient-specific three-dimensional anatomically neutral biomap to compensate for the feature anomalies.

9. The method of claim 6, wherein creating the three dimensional biomap of the patient further comprises:
obtaining a second position-specific template 3D model corresponding to a position of the patient when the medical imaging data was obtained for the patient; and
mapping voxel data associated with a second timepoint from the hierarchical virtual space data into the second position-specific template; and
creating a second three-dimensional real-time model of the patient based on mapping the voxel data associated with the second timepoint from the hierarchical virtual space data into the second position-specific template.

10. The method of claim 9, further comprising:
obtaining a three-dimensional anatomically neutral template model; and
mapping image data from the first and second three-dimensional real-time models to the three-dimensional anatomically neutral template model to create a patient-specific three-dimensional anatomically neutral biomap.

11. The method of claim 10, wherein mapping the image data comprises:
identifying a skeleton of the patient from the first three-dimensional real-time model;
locating bones and joints of the skeleton from the first three-dimensional real-time mode;
performing a geometric transform to reorient the skeleton; and
performing an elastic transform to modify soft issue orientation in accordance with reorientation of the skeleton.

12. A precision biomap computing system, comprising:
a database configured to store image data; and
a biomap creation computing unit configured to:
receive medical imaging data for a patient from one or more medical imaging data sources;
organize the medical imaging data into hierarchical virtual space data;
store the hierarchical virtual space data in the database; and
create a three dimensional biomap of the patient based on the hierarchical virtual space data.

13. The precision biomap computing system of claim 12, wherein the biomap creation computing unit is further configured to convert the three dimensional bimap to a digital twin biomap interface, and wherein the precision biomap computing system further comprises a graphical user interface configured to interactively present the digital twin biomap interface.

14. The precision biomap computing system of claim 12, wherein the biomap creation computing unit is further configured to:
convert the medical imaging data to a first real-time three dimensional model corresponding to a first timepoint;
convert second medical imaging data to a second real-time three dimensional mode corresponding to a second timepoint; and
create a three dimensional anatomically neutral model based on the first and second real-time three dimensional models.

15. The precision biomap computing system of claim 12, wherein the biomap creation computing unit is further configured to:
perform a feature recognition algorithm to identify features of the patient based on the medical imaging data; and
convert the medical imaging to voxel data, wherein the voxel data include the identified features.

16. The precision biomap computing system of claim 12, wherein the biomap creation computing unit is further configured to:
obtain a first position-specific template 3D model corresponding to a position of the patient when the medical imaging data was obtained for the patient;
map voxel data associated with a first timepoint from the hierarchical virtual space data into the first position-specific template; and
in response to the mapping, create a first three-dimensional real-time model of the patient.

17. The precision biomap computing system of claim 16, wherein the biomap creation computing unit is further configured to:
obtain a three-dimensional anatomically neutral template model; and
map image data from the first three-dimensional real-time model to the three-dimensional anatomically neutral template model to create a patient-specific three-dimensional anatomically neutral biomap.

18. The precision biomap computing system of claim 17, wherein the biomap creation computing unit is further configured to:
identify feature anomalies in the patient-specific three-dimensional anatomically neutral biomap; and
adjust the patient-specific three-dimensional anatomically neutral biomap to compensate for the feature anomalies.

19. The precision biomap computing system of claim 12, wherein the biomap creation computing unit is further configured to
identify a skeleton of the patient from the first three-dimensional real-time model;
locate bones and joints of the skeleton from the first three-dimensional real-time mode;
perform a geometric transform to reorient the skeleton; and
perform an elastic transform to modify soft issue orientation in accordance with reorientation of the skeleton.

20. A non-transitory computer-readable medium having instructions stored thereon that, upon execution, cause a computing device to perform operations comprising:
receiving medical imaging data for a patient;
organizing the medical imaging data into hierarchical virtual space data; and
creating a three dimensional biomap of the patient based on the hierarchical virtual space data.
